(19) United States
(12) Patent Application Publication
(10) Pub. No.: US 2022/0287818 A1
(43) Pub. Date: Sep. 15, 2022

(54) STENTS AND METHODS OF MAKING AND USING THE SAME

(71) Applicant: THE CORPORATION OF MERCER UNIVERSITY, Macon, GA (US)

(72) Inventors: Joanna L. Thomas, Macon, GA (US); Sagar G. Patel, Perry, GA (US)

(21) Appl. No.: 17/693,533

(22) Filed: Mar. 14, 2022

Related U.S. Application Data
(60) Provisional application No. 63/161,211, filed on Mar. 15, 2021.

(51) Int. Cl. A61F 2/04; A61F 2/82

(52) U.S. Cl. CPC A61F 2/04; A61F 2/82; A61F 2002/041

(57) ABSTRACT

Stents are disclosed. The stents provide novel stent designs that can, in some cases, span bifurcations in a duct, such as the extrahepatic bile duct (EHBD) (e.g., where the right hepatic duct meets the left hepatic duct), without impeding bile flow from either duct branch. Methods of making and using stents are also disclosed.

### CROSS-REFERENCE TO RELATED APPLICATIONS

This patent application claims the benefit of priority to U.S. Provisional Patent Application Ser. No. 63/161,211 filed on Mar. 15, 2021, and entitled “STENTS AND METHODS OF MAKING AND USING THE SAME,” the subject matter of which is hereby incorporated by reference in its entirety.

### FIELD OF THE INVENTION

The present invention is directed to stents such as gastrointestinal stents and biliary stents. The present invention is further directed to methods of making and using stents.

### BACKGROUND

The etiology of cholestasis is variable, but often it is caused by strictures in the extrahepatic bile ducts (EHBD). Nonpharmacological treatments for biliary strictures are self-expanding metal stents (SEMs) or plastic stents that are inserted during cholangiopancreatography to re-establish bile flow. Plastic stents, unlike SEMS, do not interfere with subsequent MRIs; however, if incorrectly placed, plastic stents block bile flow at bifurcations of the EHBD.

Efforts continue to further develop stents.

### SUMMARY

The present invention addresses some of the difficulties and problems in the art by the discovery of new stents. The stents described herein provide a novel stent design that can span bifurcations in the extrahepatic bile duct (EHBD) (e.g., where the right hepatic duct meets the left hepatic duct) without impeding bile flow from either duct branch. Accordingly, the present invention is directed to stents including gastrointestinal stents and biliary stents.

In one exemplary embodiment, the stent of the present invention comprises: (I) a first end tubular member comprising (i) a first end tubular member outer surface, (ii) a first end tubular member inner surface, (iii) a first end tubular member wall extending between the first end tubular member outer surface and the first end tubular member inner surface, and (iv) a first end tubular member lumen extending through the first end tubular member along the first end tubular member inner surface; (II) a second end tubular member comprising (i) a second end tubular member outer surface, (ii) a second end tubular member inner surface, (iii) a second end tubular member wall extending between the second end tubular member outer surface and the second end tubular member inner surface, and (iv) a second end tubular member lumen extending through the second end tubular member along the second end tubular member inner surface; and (III) a fluid-permeable section extending between and connecting said first end tubular member to said second end tubular member, said fluid-permeable section (a) comprising one or more struts with each strut extending between and connecting said first end tubular member to said second end tubular member, (b) allowing fluid flow from each of (i) said first end tubular member and (ii) said second end tubular member to pass into and through said fluid-permeable section to a location outside an outer boundary of said fluid-permeable section, and (c) having a compliance that is more than (i) said first end tubular member, and (ii) said second end tubular member.

In another exemplary embodiment, the stent of the present invention comprises: (I) a first end tubular member comprising (i) a first end tubular member outer surface, (ii) a first end tubular member inner surface, (iii) a first end tubular member wall extending between the first end tubular member outer surface and the first end tubular member inner surface, and (iv) a first end tubular member lumen extending through the first end tubular member along the first end tubular member inner surface; (II) a second end tubular member comprising (i) a second end tubular member outer surface, (ii) a second end tubular member inner surface, (iii) a second end tubular member wall extending between the second end tubular member outer surface and the second end tubular member inner surface, and (iv) a second end tubular member lumen extending through the second end tubular member along the second end tubular member inner surface; (III) a fluid-permeable section extending between and connecting said first end tubular member to said second end tubular member, said fluid-permeable section (a) comprising one or more struts with each strut extending between and connecting said first end tubular member to said second end tubular member, (b) allowing fluid flow from each of (i) said first end tubular member and (ii) said second end tubular member to pass into and through said fluid-permeable section to a location outside an outer boundary of said fluid-permeable section, and (c) having a compliance that is more than (i) said first end tubular member, and (ii) said second end tubular member; (IV) a third tubular member comprising (i) a third tubular member outer surface, (ii) a third tubular member inner surface, (iii) a third tubular member wall extending between the third tubular member outer surface and the third tubular member inner surface, and (iv) a third tubular member lumen extending through the third tubular member along the third tubular member inner surface; and (V) a second fluid-permeable section extending between and connecting said second tubular member to said third tubular member, said second fluid-permeable section (a) comprising one or more struts with each strut extending between and connecting said second tubular member to said third tubular member, (b) allowing fluid flow from each of (i) said second tubular member and (ii) said third tubular member to pass into and through said second fluid-permeable section to a location outside an outer boundary of said second fluid-permeable section, and (c) having a compliance that is more than (i) said second tubular member, and (ii) said third tubular member.

In yet another exemplary embodiment, the stent of the present invention comprises: (I) a first end tubular member comprising (i) a first end tubular member outer surface, (ii) a first end tubular member inner surface, (iii) a first end tubular member wall extending between the first end tubular member outer surface and the first end tubular member inner surface, and (iv) a first end tubular member lumen extending through the first end tubular member along the first end tubular member inner surface; (II) a second end tubular member comprising (i) a second end tubular member outer surface, (ii) a second end tubular member inner surface, (iii) a second end tubular member wall extending between the second end tubular member outer surface and the second end tubular member inner surface, and (iv) a second end tubular member lumen extending through the second end tubular member along the second end tubular member inner surface; (III) a fluid-permeable section extending between and connecting said first end tubular member to said second end tubular member, said fluid-permeable section (a) comprising one or more struts with each strut extending between and connecting said first end tubular member to said second end tubular member, (b) allowing fluid flow from each of (i) said first end tubular member and (ii) said second end tubular member to pass into and through said fluid-permeable section to a location outside an outer boundary of said fluid-permeable section, and (c) having a compliance that is more than (i) said first end tubular member, and (ii) said second end tubular member; (IV) a third tubular member comprising (i) a third tubular member outer surface, (ii) a third tubular member inner surface, (iii) a third tubular member wall extending between the third tubular member outer surface and the third tubular member inner surface, and (iv) a third tubular member lumen extending through the third tubular member along the third tubular member inner surface; (V) a second fluid-permeable section extending between and connecting said second tubular member to said third tubular member, said second fluid-permeable section (a) comprising one or more struts with each strut extending between and connecting said second tubular member to said third tubular member, (b) allowing fluid flow from each of (i) said second tubular member and (ii) said third tubular member to pass into and through said second fluid-permeable section to a location outside an outer boundary of said second fluid-permeable section, and (c) having a compliance that is more than (i) said second tubular member, and (ii) said third tubular member; (VI) a fourth tubular member comprising (i) a fourth tubular member outer surface, (ii) a fourth tubular member inner surface, (iii) a fourth tubular member wall extending between the fourth tubular member outer surface and the fourth tubular member inner surface, and (iv) a fourth tubular member lumen extending through the fourth tubular member along the fourth tubular member inner surface; and (VII) a third fluid-permeable section extending between and connecting said third tubular member to said fourth tubular member, said third fluid-permeable section (a) comprising one or more struts with each strut extending between and connecting said third tubular member to said fourth tubular member, (b) allowing fluid flow from each of (i) said third tubular member and (ii) said fourth tubular member to pass into and through said third fluid-permeable section to a location outside an outer boundary of said third fluid-permeable section, and (c) having a compliance that is more than (i) said third tubular member, and (ii) said fourth tubular member.

The present invention further relates to methods of making stents. In one exemplary embodiment, the method of making a stent comprises: 3D printing the herein described stent.

The present invention even further relates to methods of using stents. In one exemplary embodiment, the method of using a stent comprises positioning the herein described stent within a duct. In some desired embodiments, the method of using a stent comprises positioning the herein described stent within adjacent duct branches at a bifurcation in a duct, for example, in the extrahepatic bile duct (EHBD) (e.g., where the right hepatic duct meets the left hepatic duct) without impeding bile flow from either duct branch. The disclosed method of using a stent may be used, for example, by gastroenterologists in palliative, short-, or long-term treatments for cholestasis.

The present invention further relates to a stent CAD model, which can be used to rapidly tailor a specific stent, such as a gastrointestinal stent or a biliary stent, that precisely matches a patient's anatomy. The stent CAD model is compatible with standard plastic stent deployment apparatuses.

In addition, the herein described stent is readily modifiable. All stent dimensions can be adjusted in the CAD model to personalize a stent for unique patient anatomy. Like other plastic stents, the herein described stent is compatible with all imaging modalities.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 is a perspective frontal view of an exemplary stent of the present invention;

FIG. 2 is a right-hand view of the exemplary stent shown in FIG. 1;

FIG. 3 is a left-hand view of the exemplary stent shown in FIG. 1;

FIG. 4 is front view of the exemplary stent shown in FIG. 1;

FIG. 5 is a rear view of the exemplary stent shown in FIG. 1;

FIG. 6 is a top view of the exemplary stent shown in FIG. 1;

FIG. 7 is a bottom view of the exemplary stent shown in FIG. 1;

FIG. 8 is a cross-sectional view of the exemplary stent shown in FIG. 1 as viewed along line 8-8 shown in FIG. 2;

FIG. 9 is a cross-sectional view of the exemplary stent shown in FIG. 1 as viewed along line 9-9 shown in FIG. 2;

FIG. 10 is a cross-sectional view of the exemplary stent shown in FIG. 1 as viewed along line 10-10 shown in FIG. 2;

FIG. 11 is a cross-sectional view of the exemplary stent shown in FIG. 1 as viewed along line 11-11 shown in FIG. 2;

FIG. 12 is a perspective view of another exemplary stent of the present invention;

FIG. 13 is a cross-sectional view of the exemplary stent shown in FIG. 12 as viewed along line 13-13 shown in FIG. 12;

FIG. 14 is a cross-sectional view of the exemplary stent shown in FIG. 12 as viewed along line 14-14 shown in FIG. 12;

FIG. 15 is a side view of an exemplary 3D printing assembly for forming exemplary stents of the present invention such as the exemplary stent shown in FIG. 1;

FIG. 16 is a perspective view of another exemplary stent of the present invention;

FIG. 17 is another perspective view of the exemplary stent shown in FIG. 16;

FIG. 18 represents (i) a front view, (ii) a rear view, (iii) a right-hand view, and (iv) a left-hand view of the exemplary stent shown in FIG. 16;

FIG. 19 is a top view of the exemplary stent shown in FIG. 16;

FIG. 20 is a bottom view of the exemplary stent shown in FIG. 16;

FIG. 21 is another front view of the exemplary stent shown in FIG. 16; and

FIG. 22 is a cross-sectional view along each of lines 22-22 shown in FIG. 21.

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to stents such as gastrointestinal stents and biliary stents. The present invention is further directed to methods of making stents such as gastrointestinal stents and biliary stents. The present invention is even further directed to methods of using stents such as gastrointestinal stents and biliary stents.

The stents of the present invention are further described in the following embodiments.

### Other Embodiments

Stents

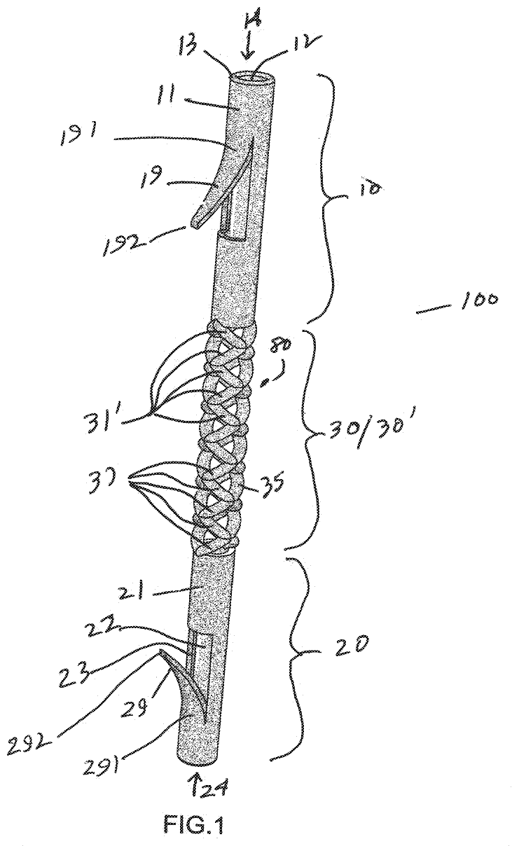

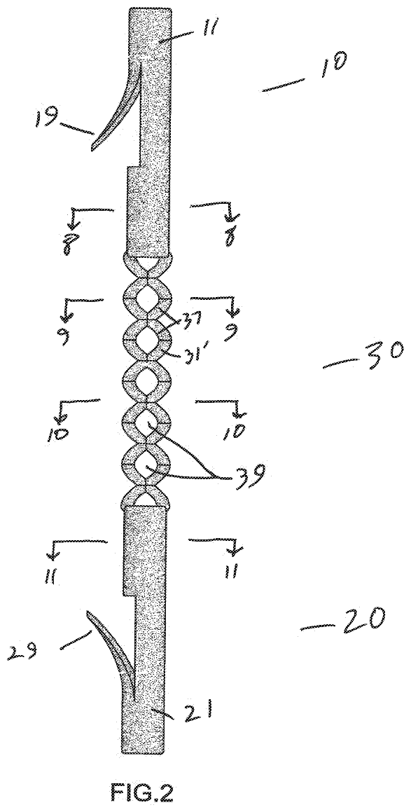

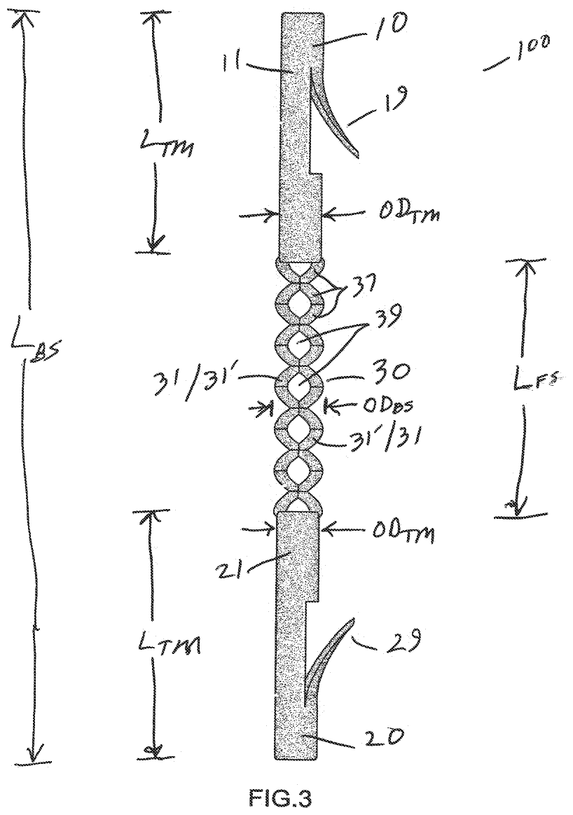

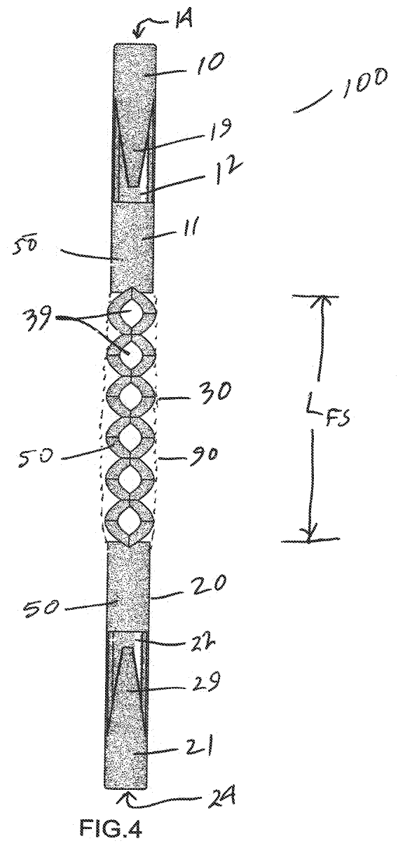

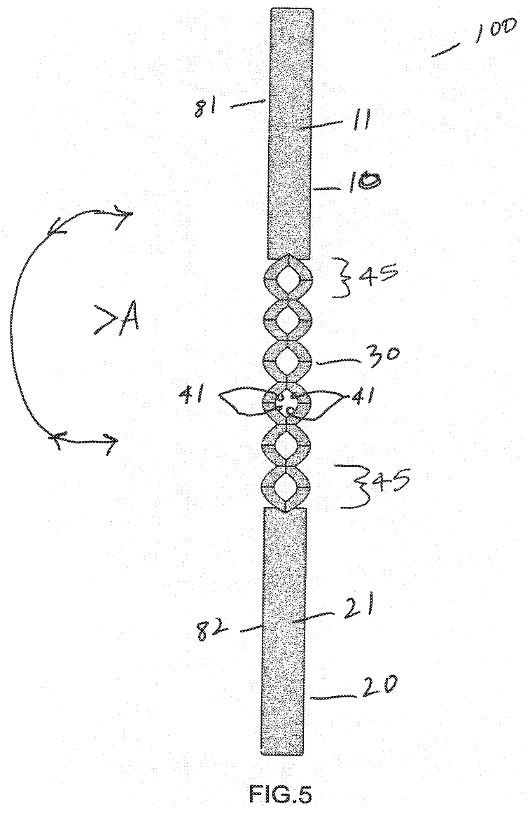

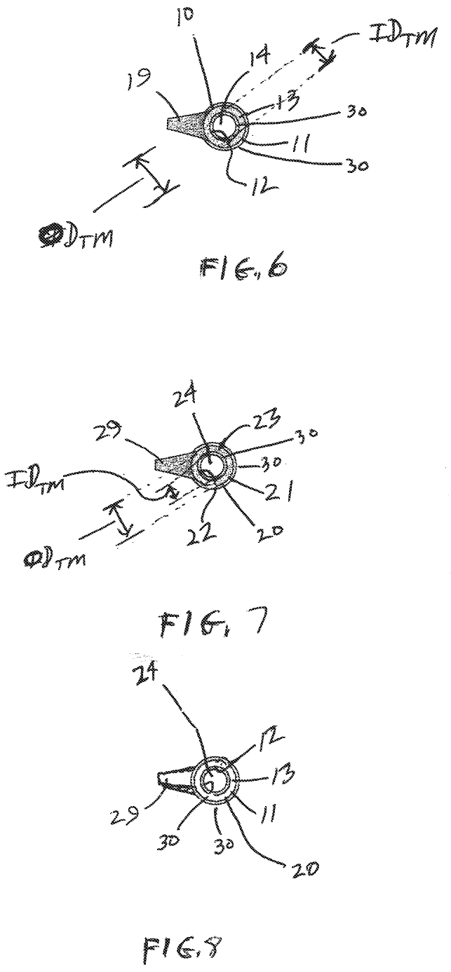

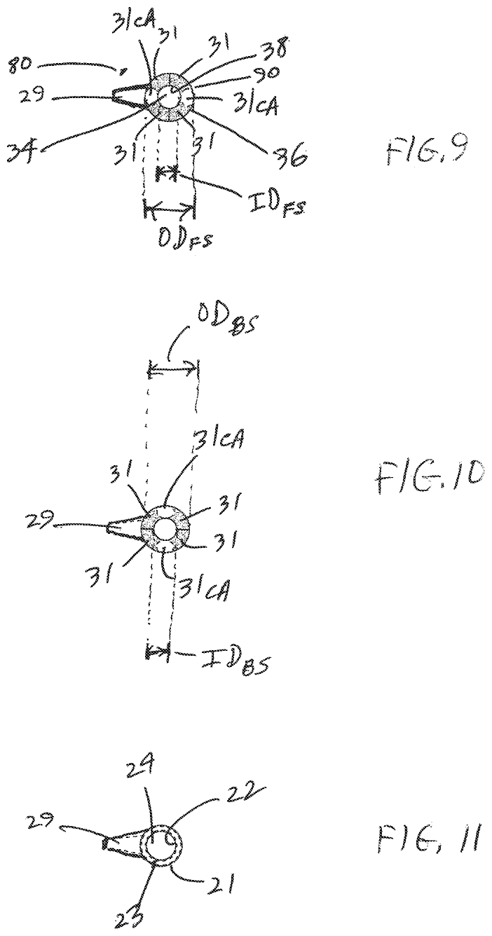

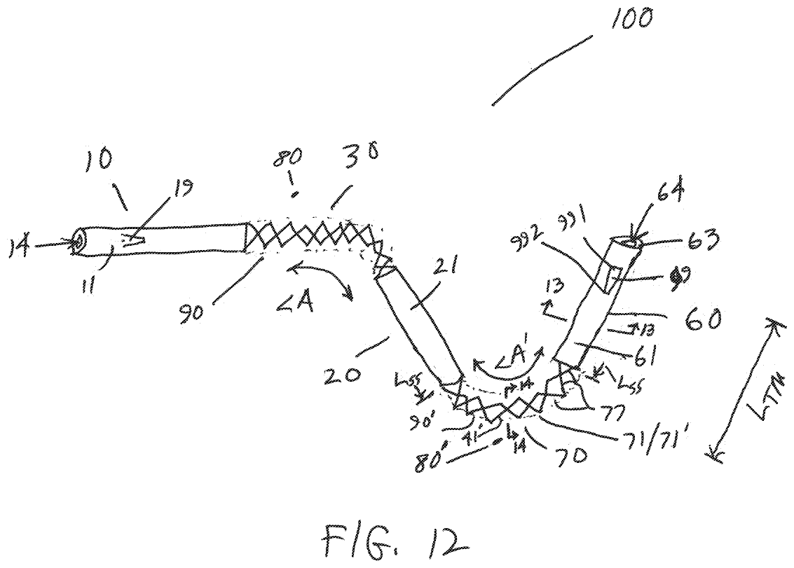

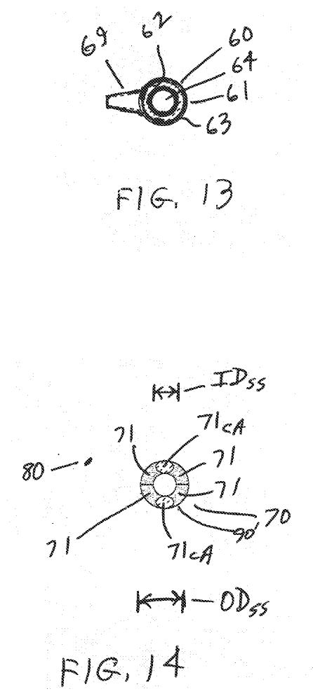

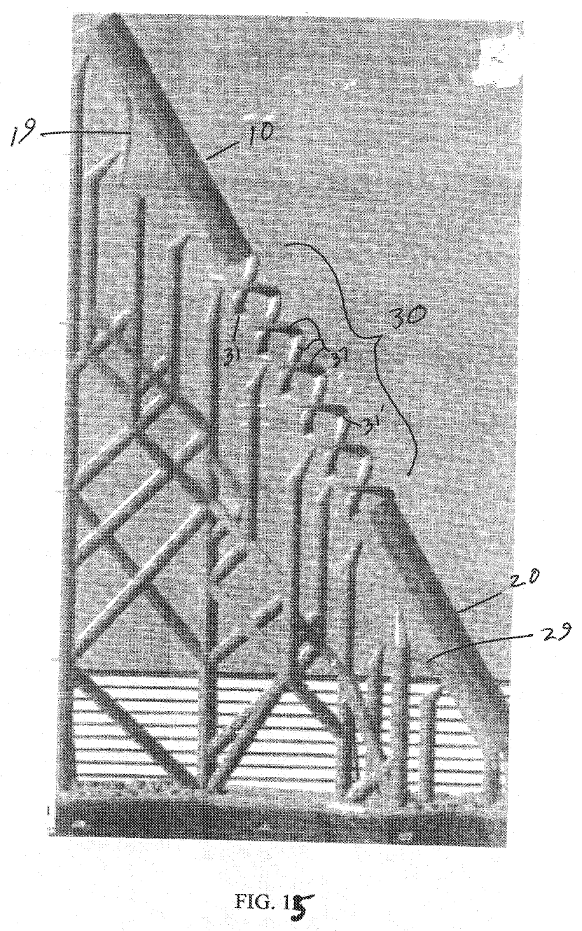

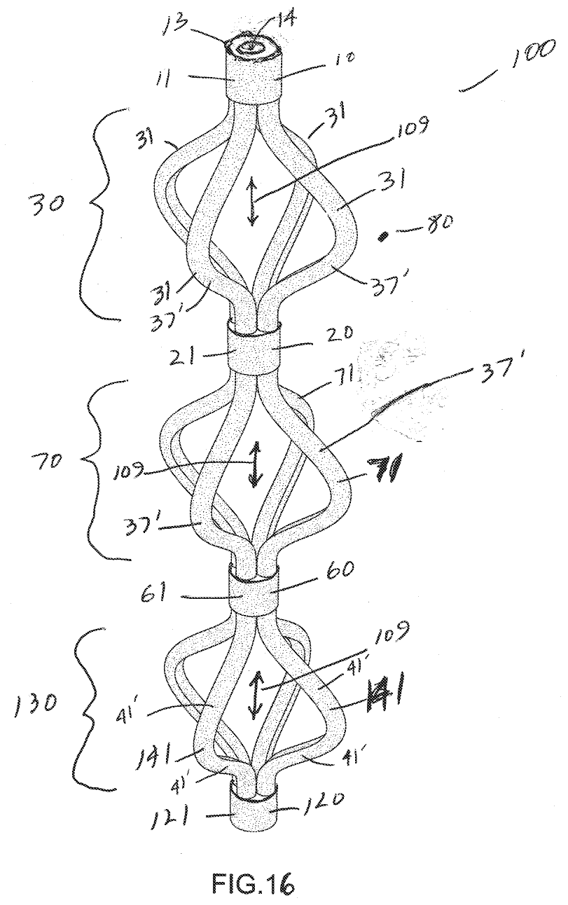

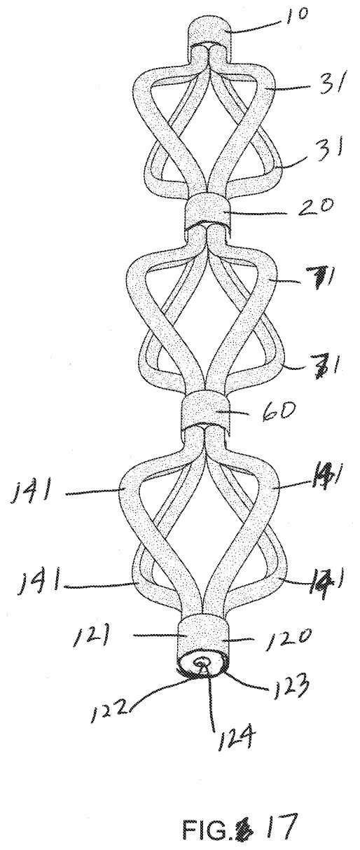

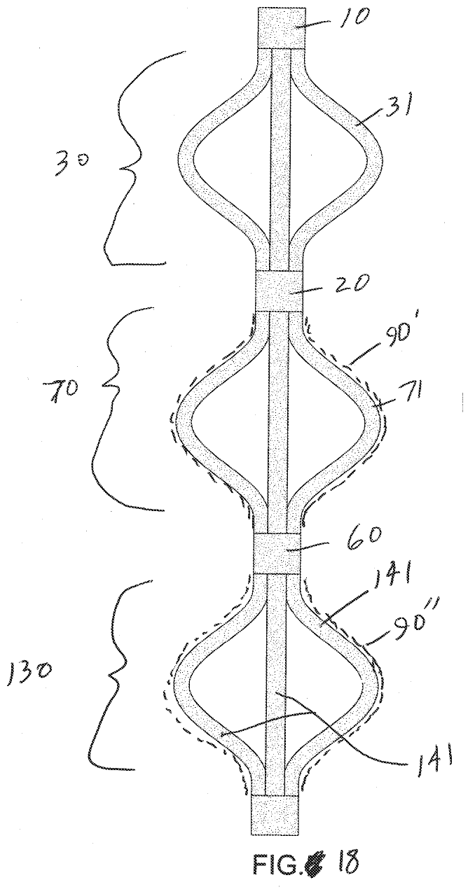

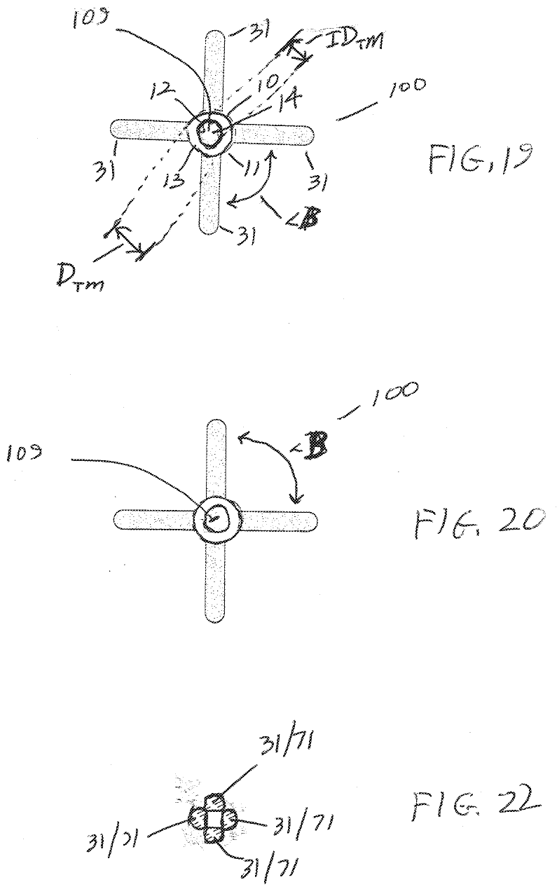

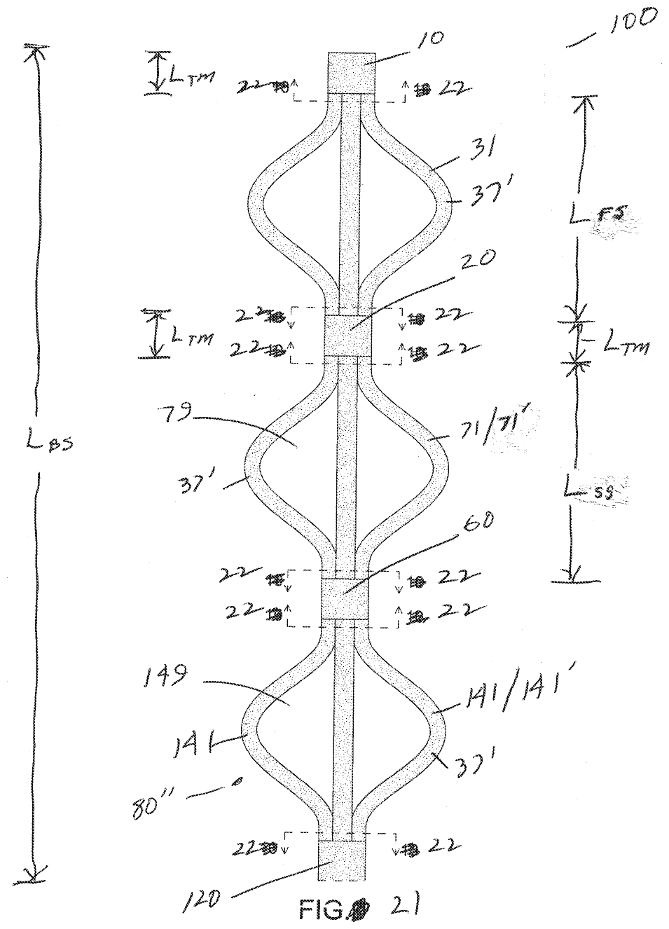

1. A stent 100 comprising: (I) a first end tubular member 10 comprising (i) a first end tubular member outer surface 11, (ii) a first end tubular member inner surface 12, (iii) a first end tubular member wall 13 extending between the first end tubular member outer surface 11 and the first end tubular member inner surface 12, and (iv) a first end tubular member lumen 14 extending through the first end tubular member 10 along the first end tubular member inner surface 12; (II) a second tubular member 20 comprising (i) a second tubular member outer surface 21, (ii) a second tubular member inner surface 22, (iii) a second tubular member wall 23 extending between the second tubular member outer surface 21 and the second tubular member inner surface 22, and (iv) a second tubular member lumen 24 extending through the second tubular member 20 along the second tubular member inner surface 22; and (III) a fluid-permeable section 30 extending between and connecting said first end tubular member 10 to said second tubular member 20, said fluid-permeable section 30 (a) comprising one or more struts 31 with each strut 31 extending between and connecting said first end tubular member 10 to said second tubular member 20, (b) allowing fluid flow from each of (i) said first end tubular member 10 and (ii) said second tubular member 20 to pass into and through said fluid-permeable section 30 to a location 80 outside an outer boundary 90 of said fluid-permeable section 30, and (c) having a compliance that is more than (i) said first end tubular member 10, and (ii) said second tubular member 20. See, for example, FIGS. 1, 12 and 16. As used herein, the term “compliance” is used to represent a rate at which an outer diameter of the fluid-permeable section 30, the first end tubular member 10, or the second tubular member 20 expands radially in response to a pressure change within the fluid-permeable section 30 or the first end tubular member 10 or the second tubular member 20. Outer boundary 90 is shown in dashed lines in FIG. 4 and is represented by a cylindrical volume (i) extending between first end tubular member 10 and said second tubular member 20, and (ii) having a volume diameter equal to or slightly larger than an outer diameter of first end tubular member 10 and/or second tubular member 20. In some preferred embodiments, the stent 100 comprises a gastrointestinal stent 100 or a biliary stent 100.
2. The stent 100 of embodiment 1, wherein each of said first end tubular member 10 and said second tubular member 20 independently has (i) a tubular member length L_{TM} of less than about 5.0 centimeters (cm), (ii) a tubular member outer diameter OD_{TM} of less than about 1.0 cm, and (iii) a tubular member inner diameter ID_{TM} of less than about 0.8 cm.
3. The stent 100 of embodiment 1 or 2, wherein each of said first end tubular member 10 and said second tubular member 20 independently has (i) a tubular member length L_{TM} of from about 0.5 cm to about 3.0 cm (or a tubular member length L_{TM} of from about 1.0 cm to about 3.0 cm), (ii) a tubular member outer diameter OD_{TM} of from about 0.3 cm to about 0.8 cm, and (iii) a tubular member inner diameter ID_{TM} of from about 0.1 cm to about 0.3 cm.
4. The stent 100 of any one of embodiments 1 to 3, wherein the one or more struts 31 have a combined strut cross-sectional area 31_{CCA} that is (i) less than a first end tubular member cross-sectional area 10_{CA}, and (ii) less than a second tubular member cross-sectional area 20_{CA}. Note that combined strut cross-sectional area 31_{CCA} equals the sum of the individual strut cross-sectional areas 31_{CCA} of the one or more struts 31. Further note that (1) each individual strut cross-sectional area 31_{CCA} of the one or more struts 31 is represented by a cross-section of material 50 (e.g., a metal, or a 3D printable polymeric material 50) used to form each strut 31 at a given location along the fluid-permeable section 30, and (2) each (i) first end tubular member cross-sectional area 10_{CA} and (ii) second tubular member cross-sectional area 20_{CA} is independently represented by a cross-section of material 50 (e.g., a metal, or a 3D printable polymeric material 50) used to form first end tubular member 10 or second tubular member 20 at a given location along first end tubular member 10 or second tubular member 20.
5. The stent 100 of any one of embodiments 1 to 4, wherein the one or more struts 31 have a combined strut cross-sectional area 31_{CCA} that (i) at least 50% less than the first end tubular member cross-sectional area 10_{CA}, and (ii) at least 50% less than the second tubular member cross-sectional area 20_{CA}.
6. The stent 100 of any one of embodiments 1 to 5, wherein each of the one or more struts 31 comprises a strand 31′ of interconnected pieces 37.
7. The stent 100 of any one of embodiments 1 to 6, wherein each of the one or more struts 31 comprises a strand 31′ of from about four to about 24 interconnected pieces 37.
8. The stent 100 of any one of embodiments 1 to 7, wherein each of the one or more struts 31 comprises a strand 31′ of about 12 interconnected pieces 37.
9. The stent 100 of any one of embodiments 1 to 8, wherein each of the one or more struts 31 has a zig-zag configuration.
10. The stent 100 of any one of embodiments 1 to 9, wherein said fluid-permeable section 30 comprising one or more section units 45 with each section unit 45 comprising eight interconnected pieces 37.
11. The stent 100 of any one of embodiments 1 to 10, wherein the one or more struts 31 form a tubular fluid-permeable section 30′, said tubular fluid-permeable section 30′ comprising (i) a tubular fluid-permeable section grid 35 extending between a tubular fluid-permeable section outer surface 36 and the tubular fluid-permeable section inner surface 38, and (iv) a tubular fluid-permeable section lumen 34 extending through the tubular fluid-permeable section 30′ along the tubular fluid-permeable section inner surface 38. See, for example, FIG. 9.
12. The stent 100 of any one of embodiments 1 to 5, wherein each of the one or more struts 31 comprises a strand 31′ comprising a single strand piece 37′.
13. The stent 100 of embodiment 12, wherein each of the single strand pieces 37′ are positioned a substantially equal distance (or an equal distance) from one another around a dissecting line 109 extending through the fluid-permeable section 30. See, for example, FIGS. 16 and 19.
14. The stent 100 of any one of embodiments 1 to 13, wherein said fluid-permeable section 30 comprising two or more struts 31 (e.g., two or more strands 31′ of interconnected pieces 37 or two single strand pieces 37′).
15. The stent 100 of any one of embodiments 1 to 14, wherein said fluid-permeable section 30 comprising from two to eight struts 31 (e.g., from two to eight strands 31′ of interconnected pieces 37 or from two to eight single strand pieces 37′).
16. The stent 100 of any one of embodiments 1 to 15, wherein said fluid-permeable section 30 comprising four struts 31 (e.g., four strands 31′ of interconnected pieces 37 or four single strand pieces 37′).
17. The stent 100 of embodiment 16, wherein each of the single strand pieces 37′ are separated from one another around dissecting line 109 by an angle B, wherein B ranges from about 75° to about 105°. See, for example, FIG. 19.
18. The stent 100 of embodiment 16 or 17, wherein each of the single strand pieces 37′ are separated from one another around dissecting line 109 by an angle B, wherein B is about 90°. See, for example, FIG. 19.
19. The stent 100 of any one of embodiments 1 to 18, wherein said fluid-permeable section 30 has (i) a fluid-permeable section length L_{FS} of less than about 5.0 cm, and (ii) a fluid-permeable section outer diameter OD_{FS} of less than about 3.0 cm (or less than about 2.5 cm, or less than about 2.0 cm, or less than about 1.5 cm, or less than about 1.0 cm). See again, for example, FIG. 9.
20. The stent 100 of any one of embodiments 1 to 11 and 14 to 19, wherein said fluid-permeable section 30 has (i) a fluid-permeable section length L_{FS} of from about 0.5 cm to about 3.5 cm, and (ii) a fluid-permeable section outer diameter OD_{FS} of from about 0.3 cm to about 0.8 cm.
21. The stent 100 of any one of embodiments 1 to 11 and 14 to 20, wherein said tubular fluid-permeable section 30 has an inner diameter ID_{FS} of less than about 0.8 cm.
22. The stent 100 of any one of embodiments 1 to 11 and 14 to 21, wherein said tubular fluid-permeable section 30 has an inner diameter ID_{FS} of from about 0.1 cm to about 0.3 cm.
23. The stent 100 of any one of embodiments 1 to 5 and 12 to 19, wherein said fluid-permeable section 30 has (i) a fluid-permeable section length L_{FS} of from about 0.5 cm to about 3.5 cm, and (ii) a fluid-permeable section outer diameter OD_{FS} of from about 0.5 cm to about 3.0 cm.
24. The stent 100 of any one of embodiments 1 to 5, 12 to 19, and 23, wherein said tubular fluid-permeable section 30 has an inner diameter ID_{FS} that varies from less than about 1.0 cm to greater than about 2.0 cm along said fluid-permeable section length L_{FS}.
25. The stent 100 of any one of embodiments 1 to 5, 12 to 19, and 23, wherein said tubular fluid-permeable section 30 has an inner diameter ID_{FS} that peaks in size at approximately a midpoint along said fluid-permeable section length L_{FS}.
26. The stent 100 of any one of embodiments 1 to 25, wherein said fluid-permeable section 30 comprises at least three fluid-permeable section openings 39 that provide fluid flow from inside said tubular fluid-permeable section 30 to location 80 outside of said tubular fluid-permeable section 30.
27. The stent 100 of any one of embodiments 1 to 11, 14 to 22 and 26, wherein said fluid-permeable section 30 comprises from about 3 fluid-permeable section openings 39 to about 48 fluid-permeable section openings 39 that provide fluid flow from said tubular fluid-permeable section lumen 34 to location 80 outside of said tubular fluid-permeable section 30.
28. The stent 100 of any one of embodiments 1 to 5, 12 to 19, 23 to 26, wherein said fluid-permeable section 30 comprises four fluid-permeable section openings 39 that provide fluid flow from inside said tubular fluid-permeable section 30 to location 80 outside of said tubular fluid-permeable section 30.
29. The stent 100 of any one of embodiments 26 to 28, wherein each fluid-permeable section opening 39 has four opening sides 41.
30. The stent 100 of any one of embodiments 1 to 29, wherein each of said first end tubular member outer surface 11 and said first end tubular member inner surface 12 is substantially continuous. As used herein, the phrase “substantially continuous” is used to describe a surface that does not have any openings or apertures therein (i.e., is fluid-impermeable) unless specifically mentioned (e.g., such as the herein-described optional flange members 19/29).
31. The stent 100 of any one of embodiments 1 to 30, wherein each of said second tubular member outer surface 21 and said second tubular member inner surface 22 is substantially continuous.
32. The stent 100 of any one of embodiments 1 to 31, wherein said first end tubular member 10 further comprises a first end tubular member flange 19 extending outward from said first end tubular member outer surface 11.
33. The stent 100 of embodiment 32, wherein said first end tubular member flange 19 comprises a first end tubular member flange connected end 191 and a first end tubular member flange engaging end 192, the first end tubular member flange engaging end 192 being closer to the fluid-permeable section 30 than said first end tubular member flange connected end 191.
34. The stent 100 of any one of embodiments 1 to 33, wherein said second tubular member 20 further comprises a second tubular member flange 29 extending outward from said second tubular member outer surface 21.
35. The stent 100 of embodiment 34, wherein said second tubular member flange 29 comprises a second tubular member flange connected end 291 and a second tubular member flange engaging end 292, the second tubular member flange engaging end 292 being closer to the fluid-permeable section 30 that said second end tubular member flange connected end 291.
36. The stent 100 of any one of embodiments 1 to 35, wherein said second tubular member 20 represents an end tubular member opposite said first tubular member 10.
37. The stent 100 of any one of embodiments 1 to 35, further comprising: (a) a third tubular member 60 comprising (i) a third tubular member outer surface 61, (ii) a third tubular member inner surface 62, (iii) a third tubular member wall 63 extending between the third tubular member outer surface 61 and the third tubular member inner surface 62, and (iv) a third tubular member lumen 64 extending through the third tubular member 60 along the third tubular member inner surface 62; and (III) a second fluid-permeable section 70 extending between and connecting said second tubular member 20 to said third tubular member 60, said second fluid-permeable section 70 (a) comprising one or more struts 71 with each strut 71 extending between and connecting said second tubular member 20 to said third tubular member 60, (b) allowing fluid flow from each of (i) said second tubular member 20 and (ii) said third tubular member 60 to pass into and through said second fluid-permeable section 70 to a location 80′ outside an outer boundary 90′ of said second fluid-permeable section 70, and (c) having a compliance that is more than (i) said second tubular member 20, and (ii) said third tubular member 60. See, for example, FIGS. 12 and 18. In FIG. 12, outer boundary 90′ is shown in dashed lines, and represents a cylindrical volume (i) extending between second tubular member 20 and third tubular member 60, and (ii) having a volume diameter equal to or slightly larger than an outer diameter of second tubular member 20 and/or third tubular member 60. In FIG. 18, outer boundary 90′ is shown in dashed lines, and represents a cylindrical volume (i) extending between second tubular member 20 and third tubular member 60, and (ii) having a volume diameter that varies from (a) being about equal to or less than an outer diameter of second tubular member 20 and/or third tubular member 60, to (b) a peak volume diameter that exceeds the outer diameter of the second tubular member 20 and the third tubular member 60 at a point along a second fluid-permeable section length L_{SS} between the second tubular member 20 and the third tubular member 60.
38. The stent 100 of embodiment 37, wherein said third tubular member 60 has (i) a tubular member length L_{TM} of less than about 5.0 cm, (ii) a tubular member outer diameter OD_{TM} of less than about 1.0 cm, and (iii) a tubular member inner diameter ID_{TM} of less than about 0.8 cm.
39. The stent 100 of embodiment 37 or 38, wherein said third tubular member 60 has (i) a tubular member length L_{TM} of from about 0.5 cm to about 3.0 cm, (ii) a tubular member outer diameter OD_{TM} of from about 0.3 cm to about 0.8 cm, and (iii) a tubular member inner diameter ID_{TM} of from about 0.1 cm to about 0.3 cm.
40. The stent 100 of any one of embodiments 37 to 39, wherein the one or more strut 71 have a combined strut cross-sectional area 71_{CCA} that is (i) less than a first end tubular member cross-sectional area 10_{CA}, and (ii) less than a second tubular member cross-sectional area 20_{CA}.
41. The stent 100 of any one of embodiments 37 to 40, wherein the one or more struts 71 have a combined strut cross-sectional area 71_{CCA} that (i) at least 50% less than the first end tubular member cross-sectional area 10_{CA}, and (ii) at least 50% less than the second tubular member cross-sectional area 20_{CA}.
42. The stent 100 of any one of embodiments 37 to 41, wherein each of the one or more struts 71 comprises a strand 71′ of interconnected pieces 77.
43. The stent 100 of any one of embodiments 37 to 42, wherein each of the one or more struts 71 comprises a strand 71′ of from about four to about 24 interconnected piece 77.
44. The stent 100 of any one of embodiments 37 to 43, wherein each of the one or more struts 71 comprises a strand 71′ of about 12 interconnected pieces 77.
45. The stent 100 of any one of embodiments 37 to 44, wherein each of the one or more struts 71 has a zig-zag configuration.
46. The stent 100 of any one of embodiments 37 to 45, wherein said second fluid-permeable section 70 comprising one or more section units 45 with each section unit 45 comprising eight interconnected pieces 77.
47. The stent 100 of any one of embodiments 37 to 46, wherein the one or more struts 71 form a second tubular fluid-permeable section 70′, said second tubular fluid-permeable section 70′ comprising (i) a second tubular fluid-permeable section grid 75 extending between a second tubular fluid-permeable section outer surface 76 and the second tubular fluid-permeable section inner surface 78, and (iv) a second tubular fluid-permeable section lumen 74 extending through the second tubular fluid-permeable section 70′ along the second tubular fluid-permeable section inner surface 38. See, for example, FIG. 12.
48. The stent 100 of any one of embodiments 37 to 41, wherein each of the one or more struts 71 comprises a strand 71′ comprising a single strand piece 37′.
49. The stent 100 of embodiment 48, wherein each of the single strand pieces 37′ are positioned a substantially equal distance (or an equal distance) from one another around a dissecting line 109 extending through the fluid-permeable section 70. See, for example, FIG. 16.
50. The stent 100 of any one of embodiments 37 to 49, wherein said second fluid-permeable section 70 comprising two or more struts 71 (e.g., two or more strands 71′ of interconnected pieces 77 or two single strand pieces 37′).
51. The stent 100 of any one of embodiments 37 to 50, wherein said second fluid-permeable section 70 comprising from two to eight struts 71 (e.g., from two to eight strands 71′ of interconnected pieces 77 or from two to eight single strand pieces 37′).
52. The stent 100 of any one of embodiments 37 to 51, wherein said second fluid-permeable section 70 comprising four struts 71 (e.g., four strands 71′ of interconnected pieces 77 or four single strand pieces 37′).
53. The stent 100 of embodiment 52, wherein each of the single strand pieces 37′ are separated from one another around dissecting line 109 by an angle B, wherein B ranges from about 75° to about 105°. See, for example, FIG. 19.
54. The stent 100 of embodiment 52 or 53, wherein each of the single strand pieces 37′ are separated from one another around dissecting line 109 by an angle B, wherein B is about 90°. See, for example, FIG. 19.
55. The stent 100 of embodiment 52 or 53, wherein each of the one or more struts 71 within said second fluid-permeable section 70 is aligned with each of the one or more struts 31 within said fluid-permeable section 30. See, for example, FIG. 16. See, for example, FIG. 16.
56. The stent 100 of any one of embodiments 37 to 55, wherein said second fluid-permeable section 70 has (i) a second fluid-permeable section length L_{SS} of less than about 5.0 cm, and (ii) a second fluid-permeable section outer diameter OD_{SS} of less than about 3.0 cm (or less than about 2.5 cm, or less than about 2.0 cm, or less than about 1.5 cm, or less than about 1.0 cm). See again, for example, FIGS. 12 and 16.
57. The stent 100 of any one of embodiments 37 to 47, 50 to 52 and 56, wherein said second fluid-permeable section 70 has (i) a second fluid-permeable section length L_{SS} of from about 0.5 cm to about 3.5 cm, and (ii) a second fluid-permeable section outer diameter OD_{SS} of from about 0.3 cm to about 0.8 cm.
58. The stent 100 of any one of embodiments 37 to 47, 50 to 52 and 56 to 57, wherein said second tubular fluid-permeable section 70 has an inner diameter ID_{SS} of less than about 0.8 cm. See again, for example, FIGS. 12 and 14.
59. The stent 100 of any one of embodiments 37 to 47, 50 to 52 and 56 to 58, wherein said second tubular fluid-permeable section 70 has an inner diameter ID_{FS} of from about 0.1 cm to about 0.3 cm.
60. The stent 100 of any one of embodiments 37 to 41, and 48 to 56, wherein said second tubular fluid-permeable section 70 has (i) a second fluid-permeable section length L_{SS} of from about 0.5 cm to about 3.5 cm, and (ii) a second fluid-permeable section outer diameter OD_{FS} of from about 0.5 cm to about 3.0 cm.
61. The stent 100 of any one of embodiments 37 to 41, 48 to 56, and 60, wherein said second tubular fluid-permeable section 70 has an inner diameter ID_{FS} that varies from less than about 1.0 cm to greater than about 2.0 cm along said second fluid-permeable section length L_{SS}.
62. The stent 100 of any one of embodiments 37 to 41, 48 to 56, and 60 to 61, wherein said second tubular fluid-permeable section 70 has an inner diameter ID_{FS} that peaks in size at approximately a midpoint along said second fluid-permeable section length L_{SS}.
63. The stent 100 of any one of embodiments 37 to 62, wherein said second fluid-permeable section 70 comprises at least three second fluid-permeable section openings 79 that provide fluid flow from inside said second tubular fluid-permeable section 70 to location 80′ outside of said second tubular fluid-permeable section 70.
64. The stent 100 of any one of embodiments 37 to 47, 50 to 52, and 63, wherein said second fluid-permeable section 70 comprises from about 3 second fluid-permeable section openings 79 to about 48 second fluid-permeable section openings 79 that provide fluid flow from inside said second tubular fluid-permeable section 70 to location 80′ outside of said second tubular fluid-permeable section 70.
65. The stent 100 of any one of embodiments 37 to 41, 48 to 52, and 60 to 64, wherein said second fluid-permeable section 70 comprises four fluid-permeable section openings 79 that provide fluid flow from inside said second tubular fluid-permeable section 70 to location 80 outside of said second tubular fluid-permeable section 70.
66. The stent 100 of any one of embodiments 63 to 65, wherein each second fluid-permeable section opening 79 has four opening sides 41′.
67. The stent 100 of any one of embodiments 37 to 66, wherein each of said third tubular member outer surface 61 and said third tubular member inner surface 62 is substantially continuous.
68. The stent 100 of any one of embodiments 37 to 67, wherein said third tubular member 60 further comprises a third tubular member flange 99 extending outward from said third tubular member outer surface 61.
69. The stent 100 of embodiment 68, wherein said third tubular member flange 99 comprises a third tubular member flange connected end 991 and a third tubular member flange engaging end 992, the third tubular member flange engaging end 992 being closer to the second fluid-permeable section 70 than said third tubular member flange connected end 991.
70. The stent 100 of any one of embodiments 37 to 69, wherein said third tubular member 60 represents an end tubular member opposite said first tubular member 10.
71. The stent 100 of any one of embodiments 37 to 69, further comprising: (a) a fourth tubular member 120 comprising (i) a fourth tubular member outer surface 121, (ii) a fourth tubular member inner surface 122, (iii) a fourth tubular member wall 123 extending between the fourth tubular member outer surface 121 and the fourth tubular member inner surface 122, and (iv) a fourth tubular member lumen 124 extending through the fourth tubular member 120 along the fourth tubular member inner surface 122; and (III) a third fluid-permeable section 130 extending between and connecting said third tubular member 60 to said fourth tubular member 120, said third fluid-permeable section 130 (a) comprising one or more struts 141 with each strut 141 extending between and connecting said third tubular member 60 to said fourth tubular member 120, (b) allowing fluid flow from each of (i) said third tubular member 60 and (ii) said fourth tubular member 120 to pass into and through said third fluid-permeable section 70 to a location 80′ outside an outer boundary 90″ of said third fluid-permeable section 130, and (c) having a compliance that is more than (i) said third tubular member 60, and (ii) said fourth tubular member 120. In FIG. 18, outer boundary 90″ is shown in dashed lines, and represents a cylindrical volume (i) extending between third tubular member 60 and fourth tubular member 120, and (ii) having a volume diameter that varies from (a) being about equal to or less than an outer diameter of the third tubular member 60 and/or the fourth tubular member 120, to (b) a peak volume diameter that exceeds the outer diameter of the third tubular member 60 and the fourth tubular member 120 at a point along a third fluid-permeable section length L_{TS} between the third tubular member 60 and the fourth tubular member 120.
72. The stent 100 of embodiment 71, wherein said fourth tubular member 120 has (i) a tubular member length L_{TM} of less than about 5.0 cm, (ii) a tubular member outer diameter OD_{TM} of less than about 1.0 cm, and (iii) a tubular member inner diameter ID_{TM} of less than about 0.8 cm.
73. The stent 100 of embodiment 71 or 72, wherein said fourth tubular member 120 has (i) a tubular member length L_{TM} of from about 1.0 cm to about 3.0 cm, (ii) a tubular member outer diameter OD_{TM} of from about 0.3 cm to about 0.8 cm, and (iii) a tubular member inner diameter ID_{TM} of from about 0.1 cm to about 0.3 cm.
74. The stent 100 of any one of embodiments 71 to 73, wherein the one or more strut 141 have a combined strut cross-sectional area 141_{CCA} that is (i) less than a third end tubular member cross-sectional area 60_{CA}, and (ii) less than a fourth tubular member cross-sectional area 120_{CA}.
75. The stent 100 of any one of embodiments 71 to 74, wherein the one or more struts 141 have a combined strut cross-sectional area 141_{CCA} that is (i) at least 50% less than the third end tubular member cross-sectional area 60_{CA}, and (ii) at least 50% less than the fourth tubular member cross-sectional area 120_{CA}.
76. The stent 100 of any one of embodiments 71 to 75, wherein each of the one or more struts 141 comprises a strand 141′ comprising a single strand piece 37′.
77. The stent 100 of embodiment 76, wherein each of the single strand pieces 37′ are positioned a substantially equal distance (or an equal distance) from one another around a dissecting line 109 extending through the third fluid-permeable section 130. See, for example, FIG. 16.
78. The stent 100 of any one of embodiments 71 to 77, wherein said third fluid-permeable section 130 comprising two or more struts 141 (e.g., two or more single strand pieces 37′).
79. The stent 100 of any one of embodiments 71 to 78, wherein said third fluid-permeable section 130 comprising from two to eight struts 141 (e.g., from two to eight single strand pieces 37′).
80. The stent 100 of any one of embodiments 71 to 79, wherein said third fluid-permeable section 130 comprising four struts 141 (e.g., four strands 141′ of four single strand pieces 37′).
81. The stent 100 of embodiment 80, wherein each of the single strand pieces 37′ are separated from one another around dissecting line 109 by an angle B, wherein B ranges from about 75° to about 105°. See, for example, FIG. 19.
82. The stent 100 of embodiment 80 or 81, wherein each of the single strand pieces 37′ are separated from one another around dissecting line 109 by an angle B, wherein B is about 90°. See, for example, FIG. 19.
83. The stent 100 of embodiment 80 or 53, wherein each of the one or more struts 141 within said third fluid-permeable section 130 is aligned with (i) each of the one or more struts 31 within said fluid-permeable section 30, and (ii) each of the one or more struts 71 within said second fluid-permeable section 70. See, for example, FIG. 16.
84. The stent 100 of any one of embodiments 71 to 83, wherein said third fluid-permeable section 130 has (i) a third fluid-permeable section length L_{SS} of less than about 5.0 cm, and (ii) a third fluid-permeable section outer diameter OD_{SS} of less than about 3.0 cm (or less than about 2.5 cm, or less than about 2.0 cm, or less than about 1.5 cm, or less than about 1.0 cm). See again, for example, FIG. 16.
85. The stent 100 of any one of embodiments 71 to 84, wherein said third tubular fluid-permeable section 130 has (i) a third fluid-permeable section length L_{SS} of from about 0.5 cm to about 3.5 cm, and (ii) a third fluid-permeable section outer diameter OD_{FS} of from about 0.5 cm to about 3.0 cm.
86. The stent 100 of any one of embodiments 71 to 85, wherein said third tubular fluid-permeable section 130 has an inner diameter ID_{FS} that varies from less than about 1.0 cm to greater than about 2.0 cm along said third fluid-permeable section length L_{SS}.
87. The stent 100 of any one of embodiments 71 to 86, wherein said third tubular fluid-permeable section 130 has an inner diameter ID_{FS} that peaks in size at approximately a midpoint along said third fluid-permeable section length L_{SS}.
88. The stent 100 of any one of embodiments 71 to 87, wherein said third fluid-permeable section 130 comprises at least three third fluid-permeable section openings 149 that provide fluid flow from inside said third tubular fluid-permeable section 130 to location 80″ outside of said third tubular fluid-permeable section 130.
89. The stent 100 of any one of embodiments 71 to 88, wherein said third fluid-permeable section 130 comprises four fluid-permeable section openings 149 that provide fluid flow from inside said third tubular fluid-permeable section 130 to location 80″ outside of said third tubular fluid-permeable section 130.
90. The stent 100 of embodiment 88 or 89, wherein each third fluid-permeable section opening 149 has four opening sides 41′.
91. The stent 100 of any one of embodiments 71 to 90, wherein each of said fourth tubular member outer surface 121 and said fourth tubular member inner surface 122 is substantially continuous.
92. The stent 100 of any one of embodiments 71 to 91, wherein said fourth tubular member 120 further comprises a fourth tubular member flange (not shown) extending outward from said fourth tubular member outer surface 121.
93. The stent 100 of embodiment 92, wherein said fourth tubular member flange comprises a fourth tubular member flange connected end (not shown) and a fourth tubular member flange engaging end (not shown), the fourth tubular member flange engaging end being closer to the third fluid-permeable section 130 than said fourth tubular member flange connected end.
94. The stent 100 of any one of embodiments 71 to 93, wherein said fourth tubular member 120 represents an end tubular member opposite said first tubular member 10. See again, for example, FIG. 16.
95. The stent 100 of any one of embodiments 1 to 94, wherein said stent 100 has (i) an overall length L_{BS} of less than about 10.0 cm, (ii) an overall outer diameter OD_{BS} of less than about 3.0 cm, and (iii) an overall inner diameter ID_{BS} of less than about 3.0 cm. In some embodiments, the overall outer diameter OD_{BS} is substantially equal to the fluid-permeable section outer diameter OD_{FS}. In some embodiments, the overall outer diameter OD_{BS} is substantially equal to the tubular member outer diameter OD_{TM}. In some embodiments, the overall inner diameter ID_{BS} is substantially equal to the fluid-permeable section inner diameter ID_{FS}. In some embodiments, the overall inner diameter ID_{BS} is substantially equal to the tubular member inner diameter ID_{TM}. In some embodiments, the fluid-permeable section inner diameter ID_{FS} is greater than the tubular member inner diameter ID_{TM}.
96. The stent 100 of any one of embodiments 1 to 95, wherein said stent 100 has (i) an overall length L_{BS} of from about 1.0 cm to about 3.0 cm, (ii) an overall outer diameter OD_{BS} of from about 0.3 cm to about 2.8 cm, and (iii) an overall inner diameter ID_{BS} of from about 0.1 cm to about 2.8 cm.
97. The stent 100 of any one of embodiments 1 to 96, wherein said stent 100 comprises a polymeric material 50.
98. The stent 100 of any one of embodiments 1 to 97 wherein said stent 100 comprises a polyolefin material 50.
99. The stent 100 of any one of embodiments 1 to 98, wherein said stent 100 comprises a polypropylene material 50.
100. The stent 100 of any one of embodiments 1 to 96, wherein said stent 100 comprises a metal material 50.
101. The stent 100 of any one of embodiments 1 to 97 and 100, wherein said stent 100 comprises a shape memory material having elastic properties such that when the fluid-permeable section 30 (and/or the first fluid-permeable section 70 and/or the third fluid-permeable section 130) is compressed from an initial configuration via an outside pressure applied onto the fluid-permeable section 30 (and/or the first fluid-permeable section 70 and/or the third fluid-permeable section 130), the fluid-permeable section 30 (and/or the first fluid-permeable section 70 and/or the third fluid-permeable section 130) returns to its initial configuration once the outside pressure is removed (i.e., not applied).
102. The stent 100 of any one of embodiments 1 to 101, wherein said stent 100 comprises a 3D printable material 50. In some embodiments, stent 100 comprises a 3D printable material 50, namely, Formlabs Durable Resin from Formlabs, Inc. (Somerville, Mass., USA).
103. The stent 100 of any one of embodiments 1 to 102, wherein said stent 100 is bendable along the fluid-permeable section 30 such that said first end tubular member 10 and said second tubular member 20 form an angle A therebetween, the angle A ranging from 0° to 180°. As shown in FIG. 5, angle A is 180°. When angle A moves closer to 0°, point 81 along first end tubular member 10 moves closer to point 82 along second tubular member 20. See again, FIG. 5.
104. The stent 100 of any one of embodiments 37 to 103, wherein said stent 100 provides unobstructed fluid flow (i) through said second tubular member lumen 24 into said second fluid-permeable section 70 and out of said second fluid-permeable section 70, and (ii) through said third tubular member lumen 74 into said second fluid-permeable section 70 and out of said second fluid-permeable section 70.
105. The stent 100 of any one of embodiments 37 to 104, wherein said stent 100 is bendable along the second fluid-permeable section 70 such that said second tubular member 20 and said third tubular member 60 form an angle A′ therebetween, the angle A′ ranging from 0° to 180°.
106. The stent 100 of any one of embodiments 37 to 105, wherein said stent 100 provides unobstructed fluid flow (i) through said second tubular member lumen 24 into said second fluid-permeable section 70 and out of said second fluid-permeable section 70, and (ii) through said third tubular member lumen 74 into said second fluid-permeable section 70 and out of said second fluid-permeable section 70.
107. The stent 100 of any one of embodiments 1 to 106, wherein said stent 100 comprises a gastrointestinal stent 100.
108. The stent 100 of any one of embodiments 1 to 107, wherein said stent 100 comprises a biliary stent 100.

Methods of Making Stents

109. A method of making the stent 100 of any one of embodiments 1 to 108, said method comprising: forming the first end tubular member 10, forming the second tubular member 20, and forming the fluid-permeable section 30.
110. The method of embodiment 109, further comprising: forming the third tubular member 60, and forming the second fluid-permeable section 70.
111. The method of embodiment 109 or 110, further comprising: forming the fourth tubular member 120, and forming the third fluid-permeable section 130.
112. The method of any one of embodiments 109 to 111, wherein said forming steps occur simultaneously.
113. The method of any one of embodiments 109 to 112, wherein said forming steps comprise: using a stent CAD model (not shown) to design a specific stent 100 that precisely matches a specific patient's anatomy (not shown).
114. The method of any one of embodiments 109 to 113, wherein said forming steps comprise a 3D printing step.
115. The method of any one of embodiments 109 to 114, wherein said forming steps comprise 3D printing a polymeric material 50 or a metal material 50.
116. The method of any one of embodiments 109 to 115, wherein said forming steps comprise 3D printing a polypropylene material 50.
117. The method of any one of embodiments 109 to 116, further comprising: washing the stent 100 in an isopropanol wash.
118. The method of any one of embodiments 109 to 117, further comprising: air drying the stent 100.
119. The method of any one of embodiments 109 to 118, further comprising: UV curing the stent 100.
120. The method of any one of embodiments 109 to 119, further comprising: sterilizing the stent 100.
121. The method of any one of embodiments 109 to 120, wherein the stent 100 comprises a gastrointestinal stent 100.
122. The method of any one of embodiments 109 to 121, wherein the stent 100 comprises a biliary stent 100.

Methods of Using Stents

123. A method of using the stent 100 of any one of embodiments 1 to 108, said method comprising: removing the stent 100 from a packaging material (not shown).
124. The method of embodiment 123, wherein said removing step takes place in an operating room setting.
125. The method of embodiment 123 or 124, further comprising: packaging the stent 100 within a packaging material (not shown).
126. The method of any one of embodiments 123 to 125, further comprising: positioning the first end tubular member 10 within a first duct (not shown), and positioning the second tubular member 20 within a second duct (not shown), wherein the first duct and the second duct are (i) different portions of the same duct, or (ii) different ducts.
127. The method of embodiment 126, wherein said positioning steps comprise: positioning the first end tubular member 10 within a first duct comprising a right hepatic duct of an extrahepatic bile duct (EHBD), and positioning the second tubular member 20 within a second duct comprising a left hepatic duct of the EHBD so as to span a bifurcation in the EHBD.
128. The method of any one of embodiments 123 to 127, further comprising: positioning the first end tubular member 10 within a first duct (not shown), positioning the second tubular member 20 within a second duct (not shown), and positioning the third tubular member 60 within a third duct (not shown), wherein the first duct, the second duct and the third duct are (i) different portions of the same duct, or (ii) two or three different ducts.
129. The method of any one of embodiments 123 to 128, wherein said method is used to treat cholestasis.

It should be understood that although the above-described stents 100, and methods are described as “comprising” one or more features, components or steps, the above-described stents 100, and methods may “comprise,” “consists of,” or “consist essentially of” any of the above-described features, components or steps of the stents 100, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a stent 100 and/or method that “comprises” a list of elements (e.g., components or steps) is not necessarily limited to only those elements (or components or steps), but may include other elements (or components or steps) not expressly listed or inherent to the stent 100 and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a stent 100 and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described stents 100, and/or methods may comprise, consist essentially of, or consist of any of the herein-described components, features, and steps, as shown in the figures with or without any feature(s) not shown in the figures. In other words, in some embodiments, the stents 100 and/or methods of the present invention do not have any additional features other than those shown in the figures, and such additional features, not shown in the figures, are specifically excluded from the stents 100 and/or methods. In other embodiments, the stents 100 and/or methods of the present invention do have one or more additional features that are not shown in the figures.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Stents 100 as described in embodiments 1 to 108 were prepared. The stents 100 were used to maintain bile flow through the extrahepatic bile duct, specifically where the right hepatic duct meets the left hepatic duct without impeding bile flow from either duct branch.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A stent 100 comprising:
(I) a first end tubular member 10 comprising (i) a first end tubular member outer surface 11, (ii) a first end tubular member inner surface 12, (iii) a first end tubular member wall 13 extending between the first end tubular member outer surface 11 and the first end tubular member inner surface 12, and (iv) a first end tubular member lumen 14 extending through the first end tubular member 10 along the first end tubular member inner surface 12;
(II) a second tubular member 20 comprising (i) a second tubular member outer surface 21, (ii) a second tubular member inner surface 22, (iii) a second tubular member wall 23 extending between the second tubular member outer surface 21 and the second tubular member inner surface 22, and (iv) a second tubular member lumen 24 extending through the second tubular member 20 along the second tubular member inner surface 22; and
(III) a fluid-permeable section 30 extending between and connecting said first end tubular member 10 to said second tubular member 20, said fluid-permeable section 30 (a) comprising one or more struts 31 with each strut 31 extending between and connecting said first end tubular member 10 to said second tubular member 20, (b) allowing fluid flow from each of (i) said first end tubular member 10 and (ii) said second tubular member 20 to pass into and through said fluid-permeable section 30 to a location 80 outside an outer boundary 90 of said fluid-permeable section 30, and (c) having a compliance that is more than (i) said first end tubular member 10, and (ii) said second tubular member 20.

2. The stent 100 of claim 1, wherein each of said first end tubular member 10 and said second tubular member 20 independently has (i) a tubular member length L_{TM} of less than about 5.0 centimeters (cm), (ii) a tubular member outer diameter OD_{TM} of less than about 1.0 cm, and (iii) a tubular member inner diameter ID_{TM} of less than about 0.8 cm.

3. The stent 100 of claim 1, wherein each of the one or more struts 31 comprises a strand 31′ of from about four to about 24 interconnected piece 37 having a zig-zag configuration.

4. The stent 100 of claim 1, wherein each of the one or more struts 31 comprises a strand 31′ comprising a single strand piece 37′, and each of the single strand pieces 37′ are positioned a substantially equal distance from one another around a dissecting line 109 extending through the fluid-permeable section 30.

5. The stent 100 of claim 1, wherein said fluid-permeable section 30 comprising four struts 31.

6. The stent 100 of claim 1, wherein said fluid-permeable section 30 comprises from about 3 fluid-permeable section openings 39 to about 48 fluid-permeable section openings 39 that provide fluid flow from inside said tubular fluid-permeable section 30 to a location 80 outside of said tubular fluid-permeable section 30, and each fluid-permeable section opening 39 has four opening sides 41.

7. The stent 100 of claim 1, wherein said first end tubular member 10 further comprises a first end tubular member flange 19 extending outward from said first end tubular member outer surface 11, and said first end tubular member flange 19 comprises a first end tubular member flange connected end 191 and a first end tubular member flange engaging end 192, the first end tubular member flange engaging end 192 being closer to the fluid-permeable section 30 than said first end tubular member flange connected end 191.

8. The stent 100 of claim 1, wherein said second tubular member 20 further comprises a second tubular member flange 29 extending outward from said second tubular member outer surface 21, and said second tubular member flange 29 comprises a second tubular member flange connected end 291 and a second tubular member flange engaging end 292, the second tubular member flange engaging end 292 being closer to the fluid-permeable section 30 that said second end tubular member flange connected end 291.

9. The stent 100 of claim 1, further comprising:
(IV) a third tubular member 60 comprising (i) a third tubular member outer surface 61, (ii) a third tubular member inner surface 62, (iii) a third tubular member wall 63 extending between the third tubular member outer surface 61 and the third tubular member inner surface 62, and (iv) a third tubular member lumen 64 extending through the third tubular member 60 along the third tubular member inner surface 62; and
(V) a second fluid-permeable section 70 extending between and connecting said second tubular member 20 to said third tubular member 60, said second fluid-permeable section 70 (a) comprising one or more struts 71 with each strut 71 extending between and connecting said second tubular member 20 to said third tubular member 60, (b) allowing fluid flow from each of (i) said second tubular member 20 and (ii) said third tubular member 60 to pass into and through said second fluid-permeable section 70 to a location 80′ outside an outer boundary 90′ of said second fluid-permeable section 70, and (c) having a compliance that is more than (i) said second tubular member 20, and (ii) said third tubular member 60.

10. The stent 100 of claim 9, wherein each of the one or more struts 71 comprises (a) a strand 71′ of from about four to about 24 interconnected piece 37 having a zig-zag configuration, or (b) a strand 71′ comprising a single strand piece 37′, and each of the single strand pieces 37′ are positioned a substantially equal distance from one another around a dissecting line 109 extending through the second fluid-permeable section 70.

11. The stent 100 of claim 9, further comprising:
(VI) a fourth tubular member 120 comprising (i) a fourth tubular member outer surface 121, (ii) a fourth tubular member inner surface 122, (iii) a fourth tubular member wall 123 extending between the fourth tubular member outer surface 121 and the fourth tubular member inner surface 122, and (iv) a fourth tubular member lumen 124 extending through the fourth tubular member 120 along the fourth tubular member inner surface 122; and
(VII) a third fluid-permeable section 130 extending between and connecting said third tubular member 60 to said fourth tubular member 120, said third fluid-permeable section 130 (a) comprising one or more struts 141 with each strut 141 extending between and connecting said third tubular member 60 to said fourth tubular member 120, (b) allowing fluid flow from each of (i) said third tubular member 60 and (ii) said fourth tubular member 120 to pass into and through said third fluid-permeable section 70 to a location 80′ outside an outer boundary 90″ of said third fluid-permeable section 130, and (c) having a compliance that is more than (i) said third tubular member 60, and (ii) said fourth tubular member 120.

12. The stent 100 of claim 1, wherein said stent 100 comprises a polymeric material 50, a metal material 50, or a shape memory material having elastic properties such that when the fluid-permeable section 30 (and/or the first fluid-permeable section 70 and/or the third fluid-permeable section 130) is compressed from an initial configuration via an outside pressure applied onto the fluid-permeable section 30 (and/or the first fluid-permeable section 70 and/or the third fluid-permeable section 130), the fluid-permeable section 30 (and/or the first fluid-permeable section 70 and/or the third fluid-permeable section 130) returns to its initial configuration once the outside pressure is removed (i.e., not applied).

13. The stent 100 of claim 1, wherein said stent 100 comprises a gastrointestinal stent 100 or a biliary stent 100.

14. A method of making the stent of claim 1, said method comprising:
forming the first end tubular member,
forming the second tubular member, and
forming the fluid-permeable section, wherein said forming steps comprise: using a stent CAD model to design a specific stent that precisely matches a specific patient's anatomy.

15. A method of using the stent of claim 1, said method comprising:
positioning the first end tubular member within a first duct comprising a right hepatic duct of an extrahepatic bile duct (EHBD), and
positioning the second tubular member within a second duct comprising a left hepatic duct of the EHBD so as to span a bifurcation in the EHBD.

16. A stent 100 comprising:
(I) a first end tubular member 10 comprising (i) a first end tubular member outer surface 11, (ii) a first end tubular member inner surface 12, (iii) a first end tubular member wall 13 extending between the first end tubular member outer surface 11 and the first end tubular member inner surface 12, and (iv) a first end tubular member lumen 14 extending through the first end tubular member 10 along the first end tubular member inner surface 12;
(II) a second tubular member 20 comprising (i) a second tubular member outer surface 21, (ii) a second tubular member inner surface 22, (iii) a second tubular member wall 23 extending between the second tubular member outer surface 21 and the second tubular member inner surface 22, and (iv) a second tubular member lumen 24 extending through the second tubular member 20 along the second tubular member inner surface 22;
(III) a fluid-permeable section 30 extending between and connecting said first end tubular member 10 to said second tubular member 20, said fluid-permeable section 30 (a) comprising one or more struts 31 with each strut 31 extending between and connecting said first end tubular member 10 to said second tubular member 20, (b) allowing fluid flow from each of (i) said first end tubular member 10 and (ii) said second tubular member 20 to pass into and through said fluid-permeable section 30 to a location 80 outside an outer boundary 90 of said fluid-permeable section 30, and (c) having a compliance that is more than (i) said first end tubular member 10, and (ii) said second tubular member 20;
(IV) a third tubular member 60 comprising (i) a third tubular member outer surface 61, (ii) a third tubular member inner surface 62, (iii) a third tubular member wall 63 extending between the third tubular member outer surface 61 and the third tubular member inner surface 62, and (iv) a third tubular member lumen 64 extending through the third tubular member 60 along the third tubular member inner surface 62; and
(V) a second fluid-permeable section 70 extending between and connecting said second tubular member 20 to said third tubular member 60, said second fluid-permeable section 70 (a) comprising one or more struts 71 with each strut 71 extending between and connecting said second tubular member 20 to said third tubular member 60, (b) allowing fluid flow from each of (i) said second tubular member 20 and (ii) said third tubular member 60 to pass into and through said second fluid-permeable section 70 to a location 80′ outside an outer boundary 90′ of said second fluid-permeable section 70, and (c) having a compliance that is more than (i) said second tubular member 20, and (ii) said third tubular member 60.

17. The stent 100 of claim 16, wherein each of the one or more struts 71 comprises a strand 71′ of from about four to about 24 interconnected piece 37 having a zig-zag configuration.

18. The stent 100 of claim 16, wherein each of the one or more struts 71 comprises a strand 71′ comprising a single strand piece 37′, and each of the single strand pieces 37′ are positioned a substantially equal distance from one another around a dissecting line 109 extending through the second fluid-permeable section 70.

19. A stent 100 comprising:
(I) a first end tubular member 10 comprising (i) a first end tubular member outer surface 11, (ii) a first end tubular member inner surface 12, (iii) a first end tubular member wall 13 extending between the first end tubular member outer surface 11 and the first end tubular member inner surface 12, and (iv) a first end tubular member lumen 14 extending through the first end tubular member 10 along the first end tubular member inner surface 12;
(II) a second tubular member 20 comprising (i) a second tubular member outer surface 21, (ii) a second tubular member inner surface 22, (iii) a second tubular member wall 23 extending between the second tubular member outer surface 21 and the second tubular member inner surface 22, and (iv) a second tubular member lumen 24 extending through the second tubular member 20 along the second tubular member inner surface 22;
(III) a fluid-permeable section 30 extending between and connecting said first end tubular member 10 to said second tubular member 20, said fluid-permeable section 30 (a) comprising one or more struts 31 with each strut 31 extending between and connecting said first end tubular member 10 to said second tubular member 20, (b) allowing fluid flow from each of (i) said first end tubular member 10 and (ii) said second tubular member 20 to pass into and through said fluid-permeable section 30 to a location 80 outside an outer boundary 90 of said fluid-permeable section 30, and (c) having a compliance that is more than (i) said first end tubular member 10, and (ii) said second tubular member 20;
(IV) a third tubular member 60 comprising (i) a third tubular member outer surface 61, (ii) a third tubular member inner surface 62, (iii) a third tubular member wall 63 extending between the third tubular member outer surface 61 and the third tubular member inner surface 62, and (iv) a third tubular member lumen 64 extending through the third tubular member 60 along the third tubular member inner surface 62;
(V) a second fluid-permeable section 70 extending between and connecting said second tubular member 20 to said third tubular member 60, said second fluid-permeable section 70 (a) comprising one or more struts 71 with each strut 71 extending between and connecting said second tubular member 20 to said third tubular member 60, (b) allowing fluid flow from each of (i) said second tubular member 20 and (ii) said third tubular member 60 to pass into and through said second fluid-permeable section 70 to a location 80′ outside an outer boundary 90′ of said second fluid-permeable section 70, and (c) having a compliance that is more than (i) said second tubular member 20, and (ii) said third tubular member 60;
(VI) a fourth tubular member 120 comprising (i) a fourth tubular member outer surface 121, (ii) a fourth tubular member inner surface 122, (iii) a fourth tubular member wall 123 extending between the fourth tubular member outer surface 121 and the fourth tubular member inner surface 122, and (iv) a fourth tubular member lumen 124 extending through the fourth tubular member 120 along the fourth tubular member inner surface 122; and
(VII) a third fluid-permeable section 130 extending between and connecting said third tubular member 60 to said fourth tubular member 120, said third fluid-permeable section 130 (a) comprising one or more struts 141 with each strut 141 extending between and connecting said third tubular member 60 to said fourth tubular member 120, (b) allowing fluid flow from each of (i) said third tubular member 60 and (ii) said fourth tubular member 120 to pass into and through said third fluid-permeable section 70 to a location 80′ outside an outer boundary 90″ of said third fluid-permeable section 130, and (c) having a compliance that is more than (i) said third tubular member 60, and (ii) said fourth tubular member 120.

20. The stent 100 of claim 19, wherein (a) said fluid-permeable section 30 comprises four struts 31, said second fluid-permeable section 70 comprises four struts 71, and said third fluid-permeable section 130 comprises four struts 141, and (b) each of the four struts 141 within said third fluid-permeable section 130 is aligned with (i) each of the four struts 31 within said fluid-permeable section 30, and (ii) each of the four struts 71 within said second fluid-permeable section 70.
